(19) United States
(12) Patent Application Publication
(10) Pub. No.: US 2024/0350274 A1
(43) Pub. Date: Oct. 24, 2024

(54) GLENOID IMPLANT AND METHOD OF INSTALLING THE SAME DURING AN ORTHOPAEDIC SHOULDER PROCEDURE

(71) Applicant: DePuy Ireland Unlimited Company, Ringaskiddy (IE)

(72) Inventors: Brian C. HODOREK, Winona Lake, IN (US); Alec CUNNINGHAM, Fort Wayne, IN (US); Colton D. MYERS, Warsaw, IN (US); Russell M. PARROTT, Winona Lake, IN (US); J. Michael WIATER, Beverly Hills, MI (US); Anand M. MURTHI, Baltimore, MD (US); Matthew J. SMITH, Columbia, MO (US); Derek J. CUFF, Venice, FL (US); Andrew JAWA, Cambridge, MA (US); Luke AUSTIN, Haddonfield, NJ (US); Matthew J. PURDY, Winona Lake, IN (US)

(21) Appl. No.: 18/636,623

(22) Filed: Apr. 16, 2024

Related U.S. Application Data
(60) Provisional application No. 63/497,695, filed on Apr. 21, 2023.

(51) Int. Cl. A61F 2/40; A61F 2/30

(52) U.S. Cl. CPC A61F 2/4081; A61F 2002/30507

(57) ABSTRACT

A glenoid implant of a shoulder prosthesis includes a baseplate having a shaft coupled to, and extending from a bottom surface, of its plate. The plate includes a peripheral ring extending downwardly away from, and circumferentially around, its bottom surface. The exterior surfaces of the peripheral ring have threads formed therein. A method of installing a glenoid implant during an orthopaedic surgical procedure is also disclosed.

The present application claims priority under 35 U.S.C. § 119 to U.S. Patent Application Ser. No. 63/497,695, filed Apr. 21, 2023, and having the title “IMPLANTS, SYSTEMS AND METHODS OF USING THE SAME,” which is herein incorporated by reference in its entirety.

### CROSS-REFERENCE TO RELATED APPLICATION

Cross reference is made to copending U.S. patent application Ser. No. ______ entitled “AUGMENTED GLENOID IMPLANT AND METHOD OF INSTALLING THE SAME DURING AN ORTHOPAEDIC SHOULDER PROCEDURE”, which is assigned to the same assignee as the present application, is filed concurrently herewith, and is hereby incorporated by reference.

### TECHNICAL FIELD

The present disclosure relates generally to orthopaedic surgery, orthopaedic implants used for replacing an articulation surface in a joint, such as shoulder prostheses. More specifically, but not exclusively, the present disclosure relates to glenoid implants for shoulder arthroplasties, as well as methods for using the same.

### BACKGROUND

Shoulder replacement, also known as shoulder arthroplasty or glenohumeral arthroplasty, is a surgical procedure in which all or part of the glenohumeral joint is replaced by a prosthetic implant. Such joint replacement surgery generally is conducted to relieve arthritis pain or fix severe physical joint damage.

In a total shoulder replacement procedure, a humeral prosthesis is used to replace the natural head of the patient's humerus. The humeral prosthesis typically includes an elongated stem component that is implanted into the intramedullary canal of the patient's humerus and a hemispherically-shaped prosthetic head component that is secured to the stem component. In such a total shoulder replacement procedure, the natural glenoid surface of the scapula is resurfaced or otherwise replaced with a glenoid component that provides a bearing surface upon which the prosthetic head component of the humeral prosthesis articulates.

However, in some cases the patient's natural shoulder, including its soft tissue, has degenerated to a severe degree of joint instability and pain. In many such cases, it may be necessary to change the mechanics of the shoulder. Reverse shoulder implants are used to do so. As its name suggests, a reverse shoulder implant reverses the anatomy, or structure, of the healthy shoulder. In particular, a reverse shoulder implant is designed such that the prosthetic head (i.e., the “ball” in the ball-and-socket joint) known as a glenosphere component is secured to the patient's scapula, with the corresponding concave bearing (i.e., the “socket” in the ball-and-socket joint) known as a humeral cup being secured to the patient's humerus. Such a reverse configuration allows the patient's deltoid muscle, which is one of the larger and stronger shoulder muscles, to raise the arm.

### SUMMARY

According to one aspect of the disclosure, a glenoid implant for implantation into a glenoid of a patient during an orthopaedic surgical procedure includes a baseplate having a plate with a top surface and a bottom surface. The plate includes a central opening extending into the plate from the top surface, and a peripheral ring extending downwardly away from, and circumferentially around, the bottom surface. The peripheral ring has an interior surface and an opposite exterior surface, with the exterior surface of the peripheral ring having a number of threads formed therein. The glenoid implant also includes a shaft coupled to and extending downwardly away from the bottom surface of the plate. The shaft has a number of threads formed in an outer surface thereof.

In an embodiment, the threads formed in the exterior surface of the peripheral ring and the threads formed in the outer surface of the shaft have a common thread pitch.

The peripheral ring may include a plurality of separate arcuate-shaped ring portions separated from one another by a gap.

The interior and exterior surfaces of the peripheral ring may have a porous structure disposed thereon.

In an embodiment, the shaft is integral with the plate.

The glenoid implant may also include a locking screw and a glenosphere. In such an embodiment, the central opening of the plate may have a number of threads formed therein. Likewise, the glenosphere may have a number of threads formed therein. The locking screw has a first number of threads configured to engage the threads of the central opening and a second number of threads configured to engage the threads of the glenosphere.

In another aspect, a glenoid implant for implantation into a glenoid of a patient during an orthopaedic surgical procedure includes a baseplate that includes a plate with a top surface and a bottom surface. The plate has a central opening extending into the plate from the top surface, and a peripheral ring extending downwardly away from, and circumferentially around, the bottom surface. The peripheral ring has an exterior surface that includes a number of threads formed therein. The glenoid implant also includes a shaft coupled to and extending downwardly away from the bottom surface of the plate. The shaft has a number of threads formed in an outer surface thereof. The threads formed in the outer surface of the shaft and the threads formed in the exterior surface of the peripheral ring have a common thread pitch.

The peripheral ring may include a plurality of separate arcuate-shaped ring portions separated from one another by a gap.

The interior and exterior surfaces of the peripheral ring may have a porous structure disposed thereon.

In an embodiment, the shaft is integral with the plate.

The glenoid implant may also include a locking screw and a glenosphere. In such an embodiment, the central opening of the plate may have a number of threads formed therein. Likewise, the glenosphere may have a number of threads formed therein. The locking screw has a first number of threads configured to engage the threads of the central opening and a second number of threads configured to engage the threads of the glenosphere.

In yet another aspect, a glenoid implant for implantation into a glenoid of a patient during an orthopaedic surgical procedure includes a baseplate that includes a plate with a top surface and a bottom surface. The plate has a central opening extending into the plate from the top surface, with the central opening of the plate having a number of threads formed therein. The plate also includes a peripheral ring extending downwardly away from, and circumferentially around, the bottom surface. The peripheral ring includes an exterior surface having a number of threads formed therein. The glenoid implant also includes a shaft coupled to and extending downwardly away from the bottom surface of the plate. The shaft has a number of threads formed in an outer surface thereof. The glenoid implant also includes a glenosphere having a number of threads formed therein, and a locking screw. The locking screw includes a first number of threads configured to engage the threads of the central opening and a second number of threads configured to engage the threads of the glenosphere.

In an embodiment, the threads formed in the outer surface of the shaft and the threads formed in the exterior surface of the peripheral ring have a common thread pitch.

The peripheral ring may include a plurality of separate arcuate-shaped ring portions separated from one another by a gap.

The interior and exterior surfaces of the peripheral ring may have a porous structure disposed thereon.

In an embodiment, the shaft is integral with the plate.

### BRIEF DESCRIPTION OF DRAWINGS

The detailed description particularly refers to the following figures, in which:

FIG. 1 is a perspective view of a baseplate of a glenoid implant;

FIG. 2 is another perspective view of the baseplate of FIG. 1;

FIG. 3 is a side view of the baseplate of FIG. 1;

FIG. 4 is another side view of the baseplate of FIG. 1;

FIG. 5 is an end view of the baseplate of FIG. 1;

FIG. 6 is another end view of the baseplate of FIG. 1;

FIG. 7 is a top view of the baseplate of FIG. 1;

FIG. 8 is a bottom view of the baseplate of FIG. 1;

FIG. 9 is a cross-sectional view of the baseplate of FIG. 1 taken along line 9-9 in FIG. 7, as viewed in the direction of the arrows;

FIG. 10 is a cross-sectional view of the baseplate of FIG. 1 taken along line 10-10 in FIG. 7, as viewed in the direction of the arrows;

FIG. 11 is a perspective view of a modular baseplate of a glenoid implant;

FIG. 12 is another perspective view of the modular baseplate of FIG. 11;

FIG. 13 is a side view of the modular baseplate of FIG. 11;

FIG. 14 is another side view of the modular baseplate of FIG. 11;

FIG. 15 is an end view of the modular baseplate of FIG. 11;

FIG. 16 is another end view of the modular baseplate of FIG. 11;

FIG. 17 is top view of the modular baseplate of FIG. 11;

FIG. 18 is a bottom view of the modular baseplate of FIG. 11;

FIG. 19 is a cross-sectional view of the modular baseplate of FIG. 11 taken along line 19-19 in FIG. 17, as viewed in the direction of the arrows;

FIG. 20 is a cross-sectional view of the modular baseplate of FIG. 11 taken along line 20-20 in FIG. 17, as viewed in the direction of the arrows;

FIG. 21 is a perspective view of another modular baseplate of a glenoid implant;

FIG. 22 is another perspective view of the modular baseplate of FIG. 21;

FIG. 23 is a side view of the modular baseplate of FIG. 21;

FIG. 24 is a top view of the modular baseplate of FIG. 21;

FIG. 25 is a side view of central posts and central screws of the modular baseplate of FIG. 21;

FIG. 26 is a perspective view of the central posts and central screws of FIG. 25;

FIG. 27 is a perspective view of a glenoid implant including the modular baseplate of FIG. 11;

FIG. 28 is a cross-sectional side view of the glenoid implant of FIG. 27 taken along line 28-28 in FIG. 27, as viewed in the direction of the arrows;

FIG. 29 is an exploded perspective view of the glenoid implant of FIG. 27; and

FIG. 30 is a side view of the glenoid implant of FIG. 27.

### DETAILED DESCRIPTION OF THE DRAWINGS

Generally stated, disclosed herein are glenoid baseplate implants for shoulder prostheses. Further, surgical methods for using the glenoid baseplate implants are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in the first figure of each embodiment.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the lower extremity, and more specifically, with the bones of the ankle, foot, and leg.

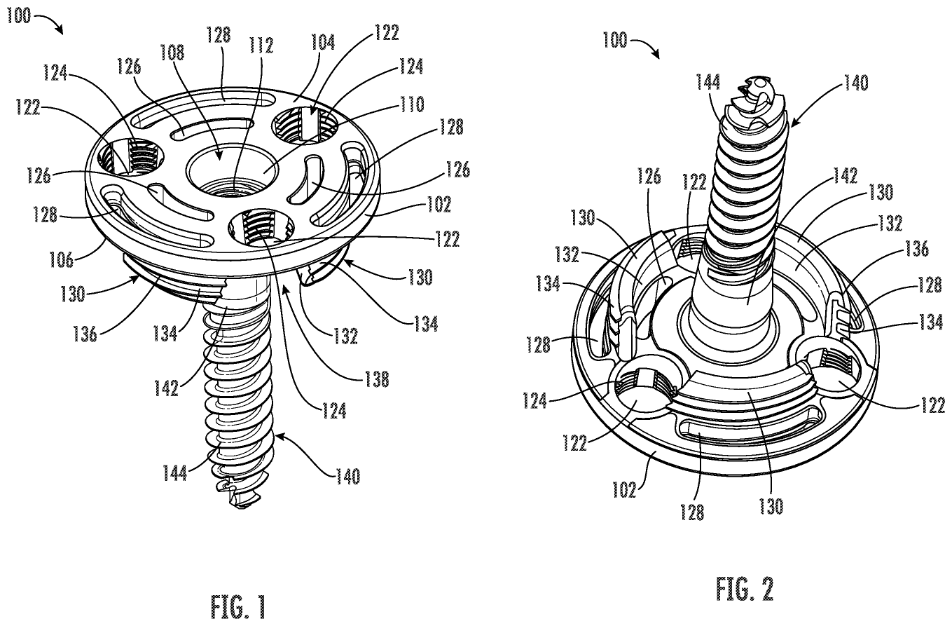

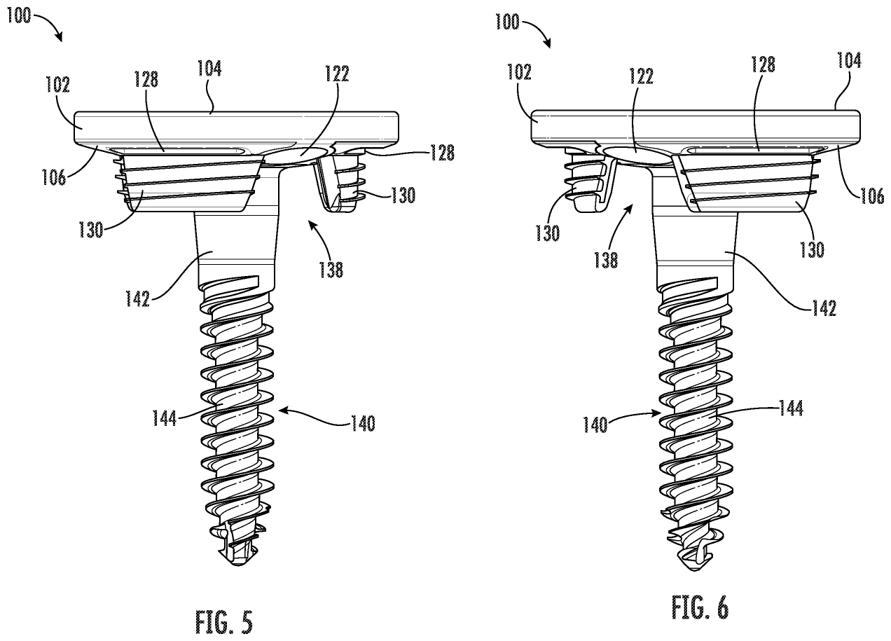

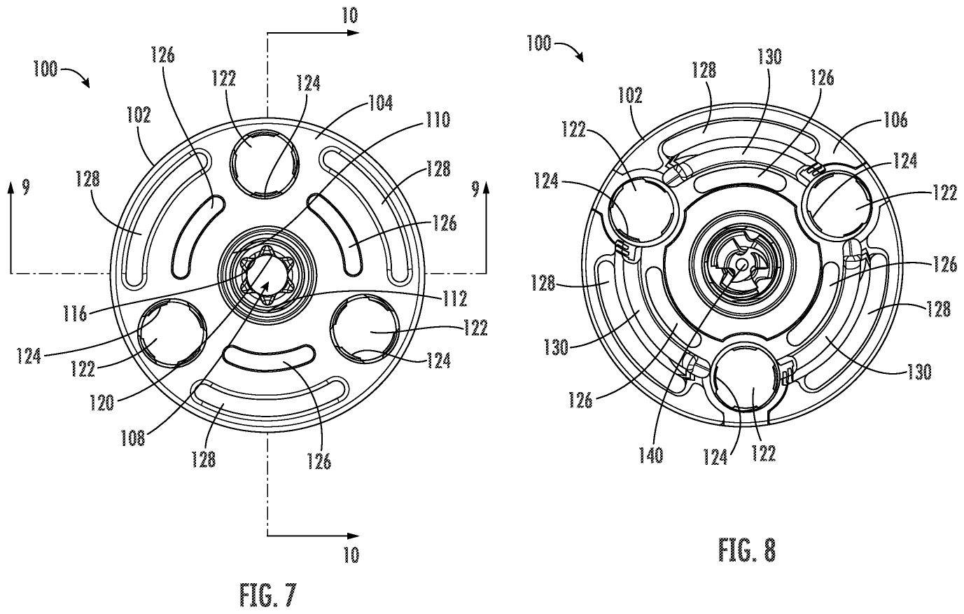

Referring to the drawings, wherein like reference numerals are used to indicate like or analogous components throughout the several views, and with particular reference to FIGS. 1-10, there is illustrated an embodiment of a baseplate 100 for a glenoid implant system or reverse glenoid implant system. The baseplate 100 may have a plate 102 and a shaft 140 coupled to and extending away from a bottom surface 106 of the base plate 100, as shown in FIGS. 1-10. The plate 102 and shaft 140 are integrally connected.

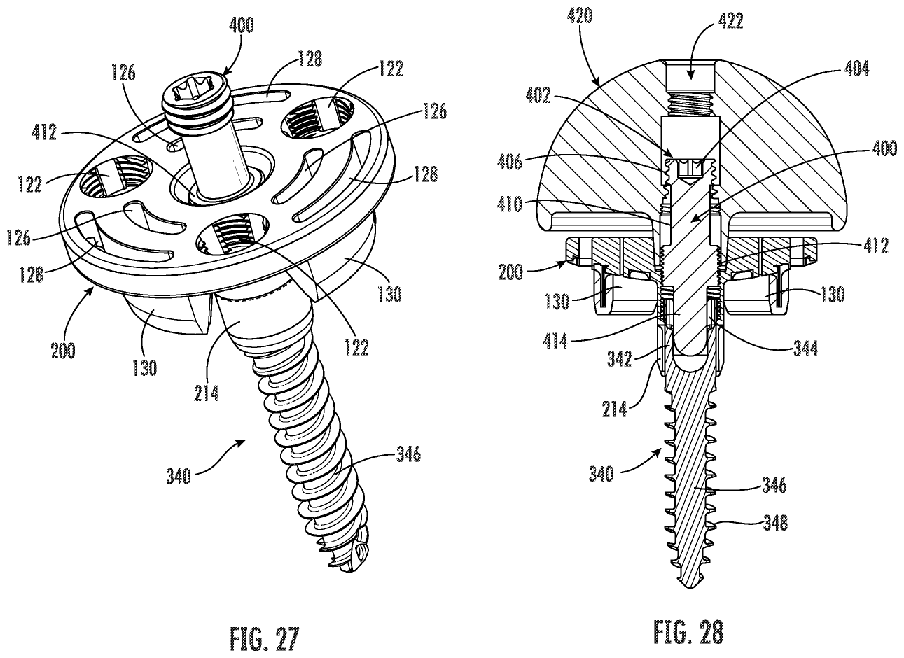

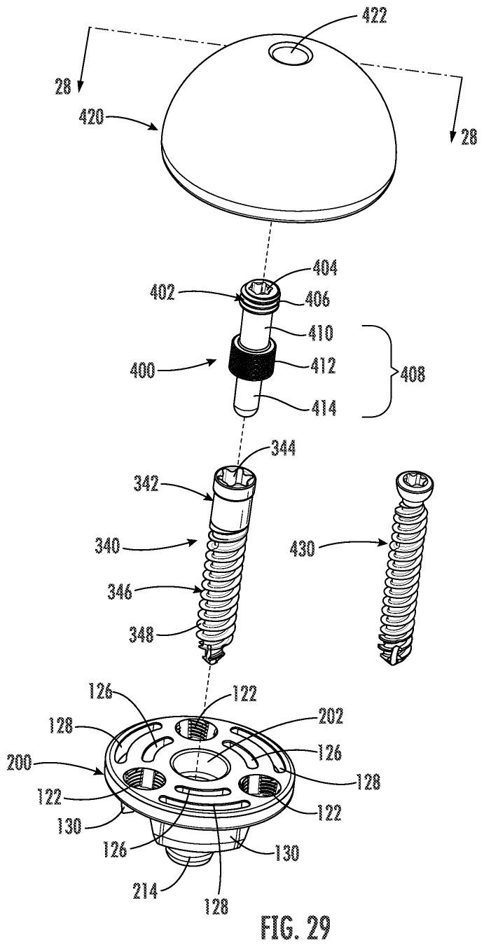

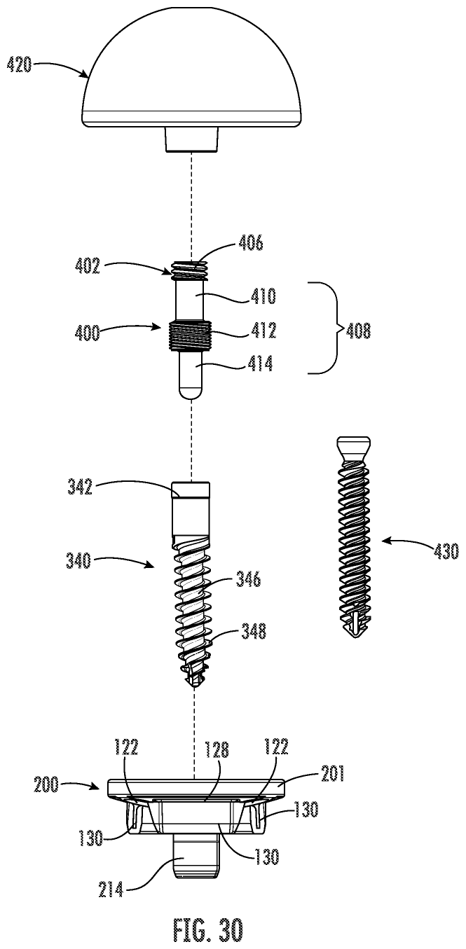

The plate 102 may have a top surface 104 and a bottom surface 106 opposite the top surface 104. The plate 102 may have, for example, a cylindrical shape with a circular top surface 104 and a circular bottom surface 106. The plate 102 may also include a central opening 108 extending from the top surface 104 through the plate 102 and into a portion of the shaft 140. The central opening 108 may be positioned, for example, in a central region of the plate 102. The central opening 108 may be, for example, configured or sized and shaped to receive a portion of a locking screw, as shown in FIGS. 27-30.

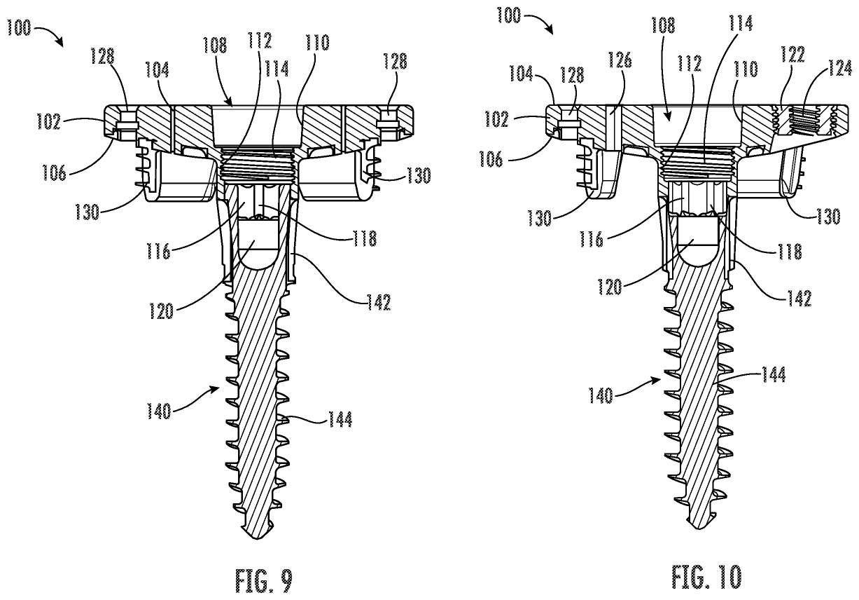

As shown in FIGS. 9-10, the central opening 108 may include a first portion 110, a second portion 112, a third portion 116, and a fourth portion 120. The first portion 110 may extend from the top surface 104 of the plate 102 toward the bottom surface. The second portion 112 may include threads 114. The threads 114 may be, for example, configured or sized and shaped to correspond to threads of the locking screw, as shown in FIGS. 27-30. The second portion 112 extends from the first portion 110 and into a portion of the shaft 140. The third portion 116 extends from the second portion 112 and further into the shaft 140. The third portion 116 may include a plurality of protrusions 118 positioned circumferentially around the interior surface of the third portion 116. The fourth portion 120 extends from the third portion 116 further into the shaft 140 and to the bottom of the central opening 108. The first portion 110 has a first diameter, the second portion 112 has a second diameter, the third portion 116 has a third diameter, and the fourth portion 120 has a fourth diameter. The first diameter may be larger than the second, third and fourth diameters. The second diameter may be larger than the third and fourth diameter. The third diameter may be larger than the fourth diameter. In addition, the fourth diameter may be smaller than the first, second and third diameter. The first portion 110 may be, for example, configured or sized and shaped to receive a portion of a glenosphere, such as glenosphere 420, as shown in FIGS. 27-30 and discussed in greater detail below. The second portion 112 may be, for example, configured or sized and shaped to threadingly engage the threaded portion of the locking screw, such as locking screw 400, as shown in FIGS. 27-30 and discussed in greater detail below. The third portion 116 and fourth portion 120 may be, for example, configured or sized and shaped to receive a distal portion of the locking screw, such as locking screw 400.

The baseplate 100 may also include screw holes or holes 122, for example, three screw holes 122 equally spaced around the circumference of the plate 102. The screw holes 122 may extend through the plate 102 from the top surface 104 to the bottom surface 106. The screw holes 122 may have, for example, threads 124 along at least a portion of the interior surface of the screw holes 122. The screw holes 122 are configured or sized and shaped to receive a bone screw for anchoring the baseplate 100 to a bone. A head of the bone screw may be seated within the screw hole 122 of the base plate 100. The plate 102 may further include at least one relief or arcuate slot 126, 128 extending through the plate 102 from the top surface 104 to the bottom surface 106. The at least one relief 126, 128 may include at least one first or interior relief 126 and at least one second or exterior relief 128. The at least one first relief 126 may be, for example, three first reliefs 126 positioned circumferentially around the central opening 108 between the screw holes 122. The at least one second relief 128 may be, for example, three second reliefs 128 positioned circumferentially around the plate 102 between the screw holes 122. The first reliefs 126 are spaced apart from the second reliefs 128. The first reliefs 126 are positioned adjacent to the central opening 108. The second relief 128 may be positioned near an exterior surface of the plate 102.

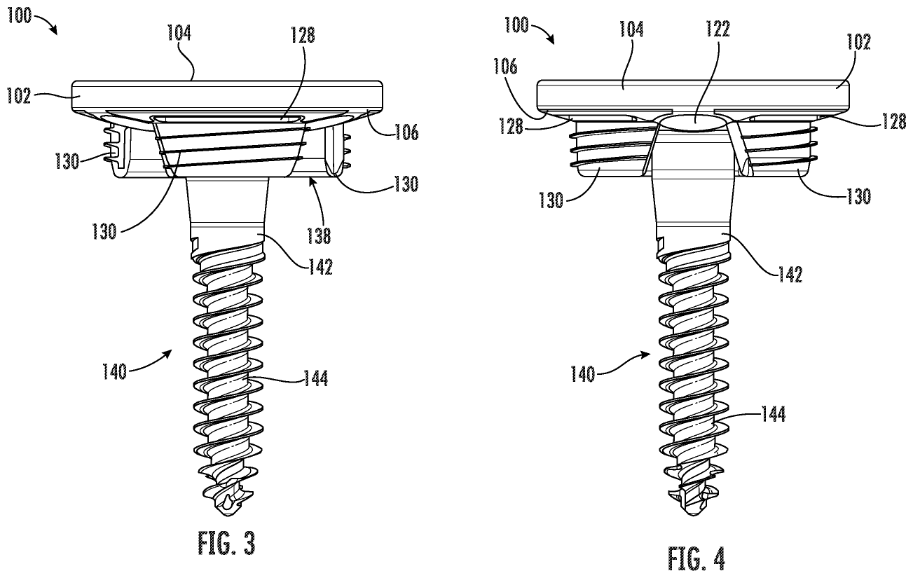

The plate 102 may include a peripheral ring illustratively in the form of, for example, at least one peripheral ring portion 130 extending away from the bottom surface 106. As shown in FIGS. 2 and 8, the peripheral ring may take the form of, for example, three arcuate-shaped peripheral ring portions 130 positioned circumferentially around the bottom surface 106. The peripheral ring portions 130 may be, for example, the same or different sizes. The peripheral ring portions 130 may be, for example, separated by a gap or space 138. The peripheral ring portions 130 may each include an interior surface 132 and an exterior surface 134. The interior surface 132 may be, for example, a uniform surface, while the exterior surface 134 may have, for example, threads 136 extending around the exterior surface 134. As can be seen in FIGS. 3-6, the threads 136 formed in the exterior surface 134 of the peripheral ring have a common thread pitch with the threads formed in the threaded portion 144 of the shaft 140. The interior and exterior surfaces 132, 134 may have, for example, a porous structure allowing for bone ingrowth. The porous structure may be, for example, 3D printed. The porous structure may be, for example, a porous metal structure or porous coating. The at least one peripheral ring portion 130 may extend away from the bottom surface 106 of the plate 102 between the first reliefs 126 and the second reliefs 128. The peripheral ring portions 130 may be inserted to engage a prepared glenoid cavity. The peripheral ring portions 130 may be, for example, arcuate and follow the same profile as the first and second reliefs or slots 126, 128. The peripheral ring portions 130 may be, for example, positioned adjacent to and between the first and second slots 126, 128. In addition, the screw holes 122 in the plate 102 may be positioned between the peripheral ring portions 130 where the spaces 138 are positioned.

The shaft 140 of the baseplate 100 may have a non-threaded portion 142 and a threaded portion 144. The non-threaded portion 142 may be coupled to and extend from the bottom surface 106 of the plate 102. The threaded portion 144 may extend from the second end of the shaft 140 to the non-threaded portion 142. The shaft 140 may extend away from the bottom surface 106 of the plate 102 near a midpoint of the plate 102. The shaft 140 may be positioned opposite the central opening 108 extending into the top surface 104, through the plate 102, and into a portion of the shaft 140. The central opening 108 may extend into at least a portion of the non-threaded portion 142. The shaft 140 may be, for example, longer than the at least one peripheral ring portions 130. The shaft 140 may extend away from the bottom surface 106 of the plate 102 between the at least one peripheral ring portions 130. The non-threaded portion 1442 may be a porous structure, which may be, for example, 3D printed. The porous structure of the non-threaded portion 142 may assist with bone ingrowth. The porous structure may be, for example, a porous metal structure or porous coating.

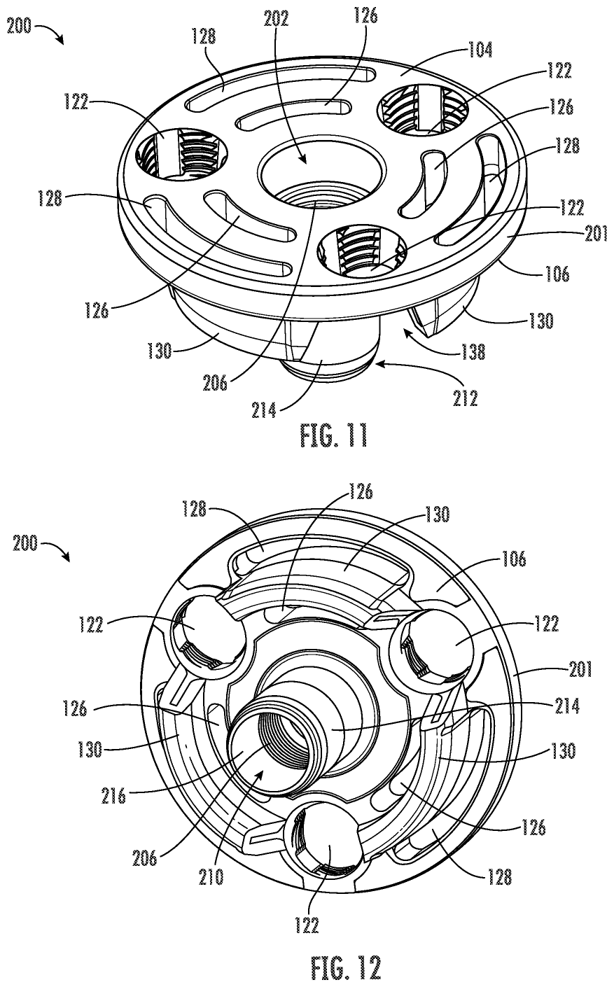

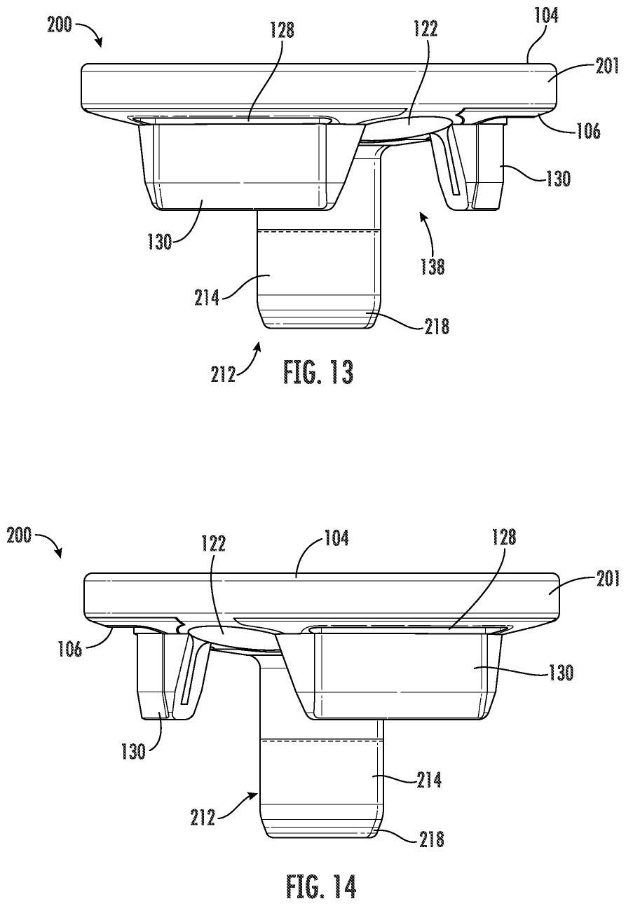

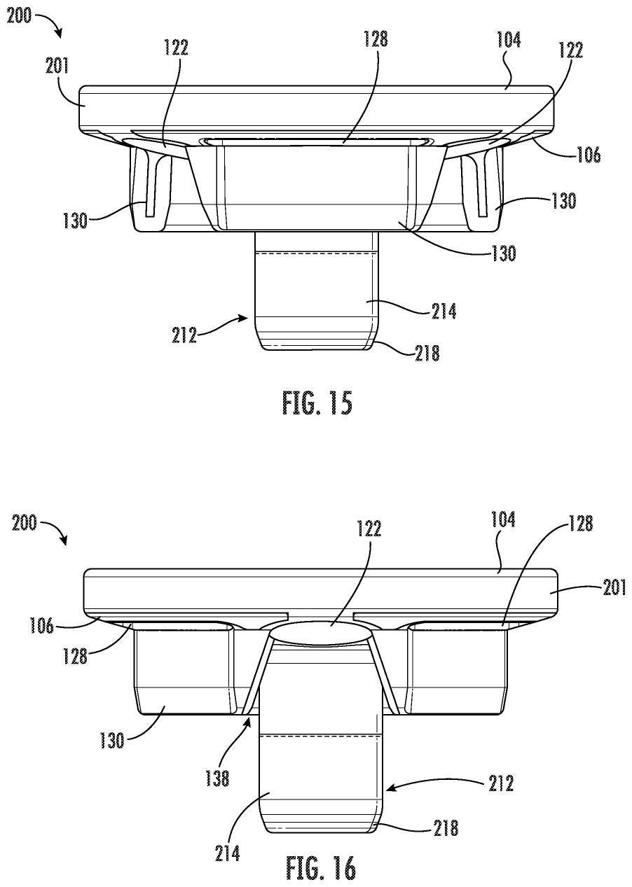

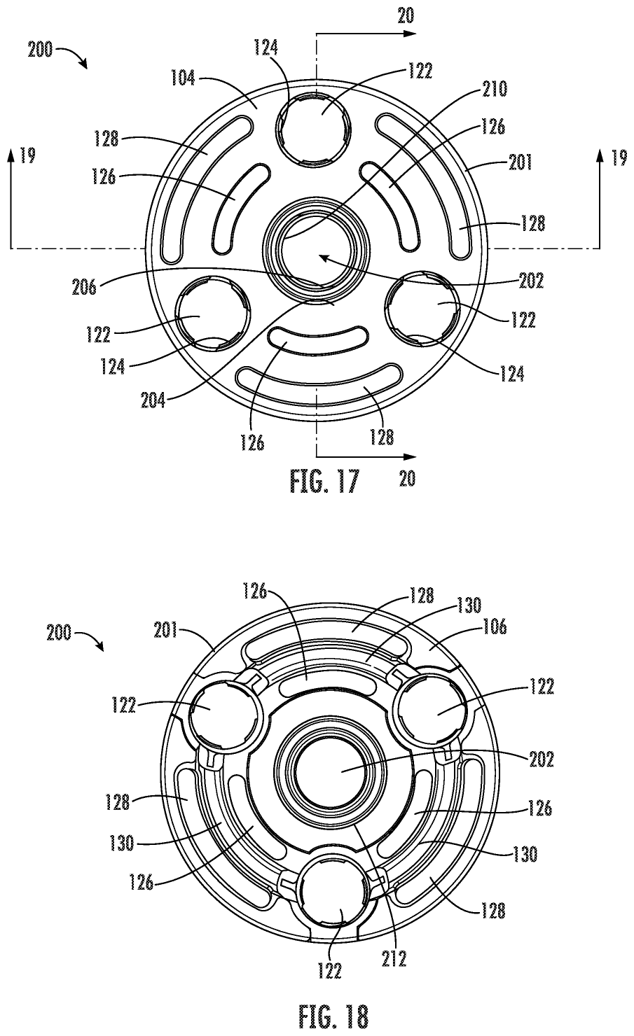

Referring now to FIGS. 11-20, another baseplate 200 for a glenoid implant system or reverse glenoid implant system. The baseplate 200 may have a plate 201, a shaft 212 coupled to and extending away from a bottom surface 106 of the baseplate 200, and a post 300, 310 or screw 320, 330, 340, 350, as shown in FIGS. 11-20. The plate 201 and shaft 212 are integrally connected, while the post 300, 310 or screw 320, 330, 340, 350 are modular or separate from the plate 201 and shaft 212.

The plate 201 may be similar to plate 102 with a central opening 202. Specifically, the plate 201 includes screw holes 122, slots 126, 128, and at least one peripheral ring portion 130, as described in greater detail above with respect to plate 102, which is described in greater detail above and which is incorporated herein. The central opening 202 extends from the top surface 104 through the plate 201 and into a portion of the shaft 212. The central opening 202 may be positioned, for example, in a central region of the plate 201. The central opening 202 may be, for example, configured or sized and shaped to receive a portion of a locking screw, as shown in FIGS. 27-30.

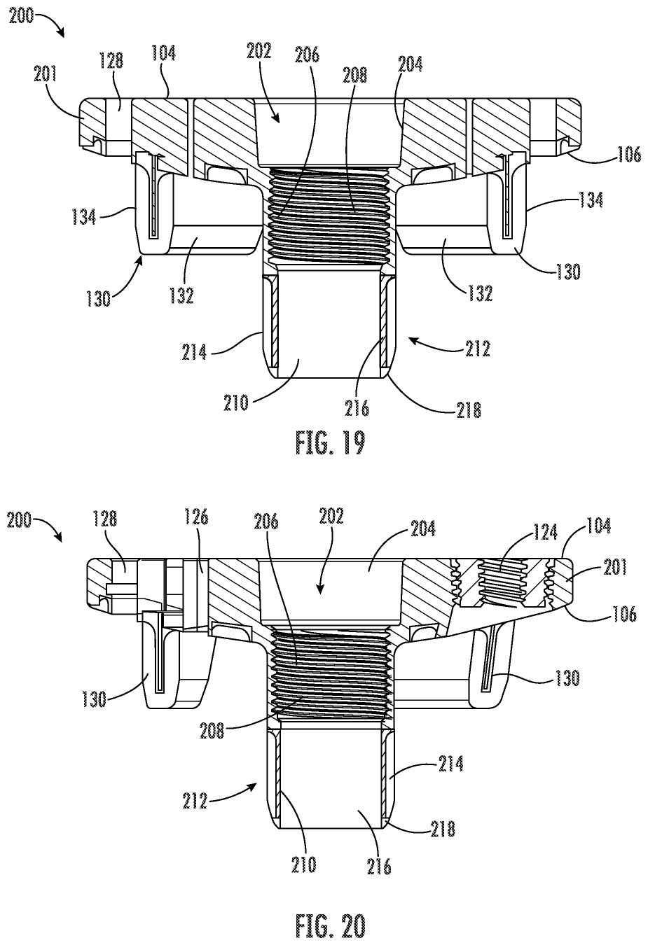

As shown in FIGS. 19-20, the central opening 202 may include a first portion 204, a second portion 206, and a third portion 210. The first portion 204 may extend from the top surface 104 of the plate 201 toward the bottom surface of the baseplate 200. The second portion 206 may include threads 208. The threads 208 may be, for example, configured or sized and shaped to correspond to threads of the locking screw, as shown in FIGS. 27-30. The second portion 206 extends from the first portion 204 and into a portion of the shaft 212. The third portion 210 extends from the second portion 206 and further into the shaft 212. The central opening 202 extends through the baseplate 200 from the top surface 104 to the bottom of the shaft 212. The first portion 204 has a first diameter, the second portion 206 has a second diameter, and the third portion 210 has a third diameter. The first diameter may be larger than the second and third diameters. The second diameter may be larger than the third diameter. The third diameter may be smaller than the first and second diameters. The first portion 204 may be, for example, configured or sized and shaped to receive a portion of a glenosphere. The second portion 206 may be, for example, configured or sized and shaped to threadingly engage the threaded portion of the locking screw. The third portion 210 may be, for example, configured or sized and shaped to receive a post 300, 310 or screw 320, 330, 340, 350 and a distal portion of the locking screw. The distal portion of the locking screw may be received within the opening 304, 314, 324, 334, 344, 354 of the post 300, 310 or screw 320, 330, 340, 350, respectively.

The shaft or stem 212 includes an exterior surface 214, an interior surface 216, and a tapered edge 218 extending between the exterior surface 214 and the interior surface 216. The exterior surface 214 may have, for example, a porous structure on at least a portion of the length allowing for bone ingrowth. The porous structure may be, for example, 3D printed. The porous structure may be, for example, a porous metal structure or porous coating. The interior surface 216 of the shaft 212 or third portion 210 of the central opening 202 receives a portion of the head 302, 312, 322, 332, 342, 352 of the post 300, 310 or screw 320, 330, 340, 350, respectively.

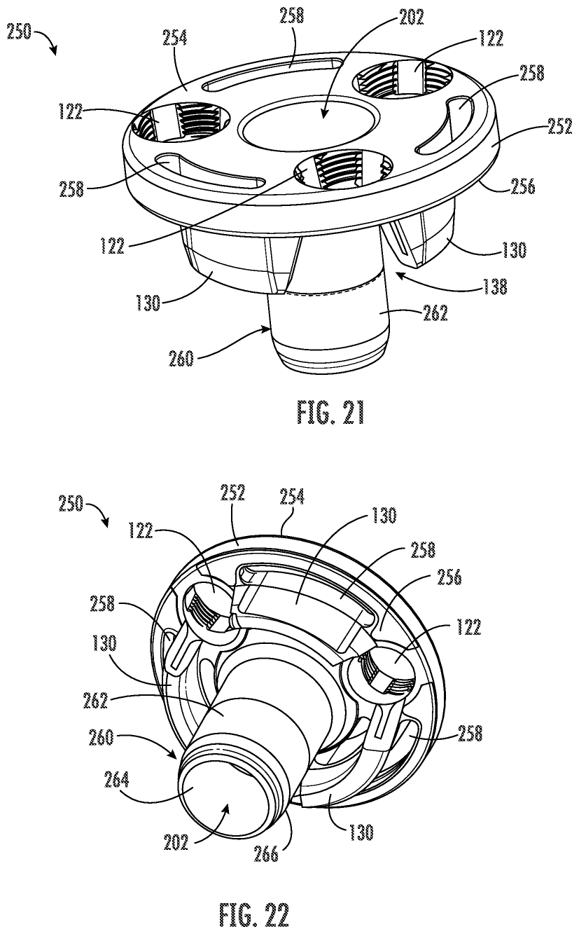

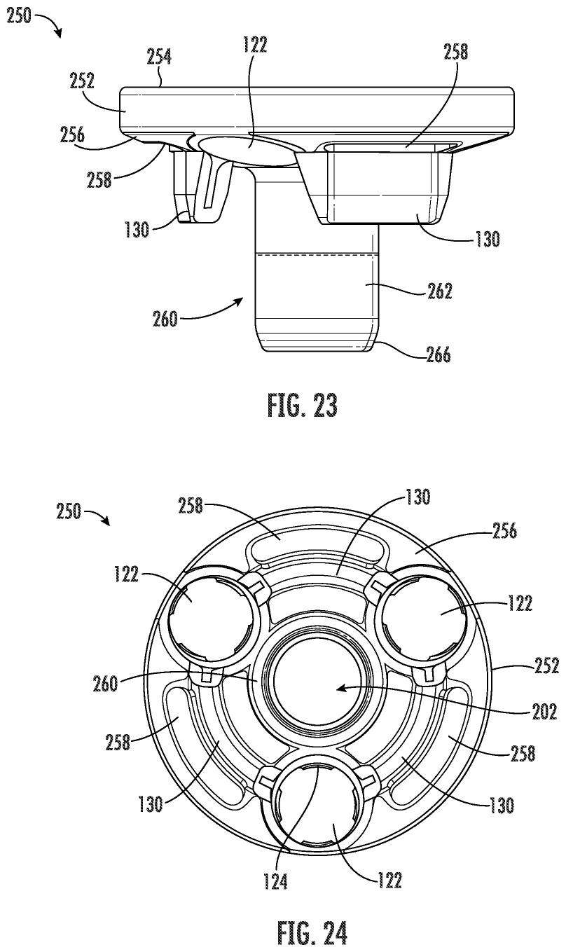

Referring now to FIGS. 21-24, another baseplate 250 for a glenoid implant system or reverse glenoid implant system. The baseplate 250 may have a plate 252 and a shaft 260 coupled to and extending away from a bottom surface 256 of the baseplate 250. The implant system includes the baseplate 250 and a post 300, 310 or screw 320, 330, 340, 350. The plate 252 and shaft 260 are integrally connected, while the post 300, 310 or screw 320, 330, 340, 350 are modular or separate from the plate 252 and shaft 260. The baseplate 250 is smaller than the baseplate 200. In addition, the plate 252 is smaller than the plate 201.

The plate 252 includes a top surface 254 and a bottom surface 256. The plate 252 also includes at least one relief or arcuate slot 258 positioned circumferentially around the plate 252. The at least one relief 258 may be, for example, three reliefs 258. The reliefs 258 extend from the top surface 254 through to the bottom surface 256 of the plate 252. The reliefs 258 may be extend through the bottom surface 256 adjacent to the exterior surface 134 of the peripheral ring portions 130. The plate 252 further includes holes 122 positioned between the reliefs 258 circumferentially around the plate 252. In addition, the plate 252 includes at least one peripheral ring portion 130 extending away from the bottom surface 256 of the plate 252. The peripheral ring portions 130 of plate 252 may have an exterior surface 134 that is, for example, not threaded. The exterior surface 134 of plate 252 may be, for example, a porous structure. Further, the interior surface 132 of plate 252 may be, for example, a porous structure. The porous structure may be, for example, a porous metal structure or porous coating that may formed by, for example, 3D printing.

The shaft or stem 260 includes an exterior surface 262, an interior surface 264, and a tapered edge 266 extending between the exterior surface 262 and the interior surface 264. The exterior surface 262 may have, for example, a porous structure on at least a portion of the length allowing for bone ingrowth. The porous structure may be, for example, 3D printed. The porous structure may be, for example, a porous metal structure or porous coating. The interior surface 264 of the shaft 260 or third portion 210 of the central opening 202 receives a portion of the head 302, 312, 322, 332, 342, 352 of the post 300, 310 or screw 320, 330, 340, 350, respectively. The shaft 260 may be, for example, the same size, larger, or smaller than the shaft 212.

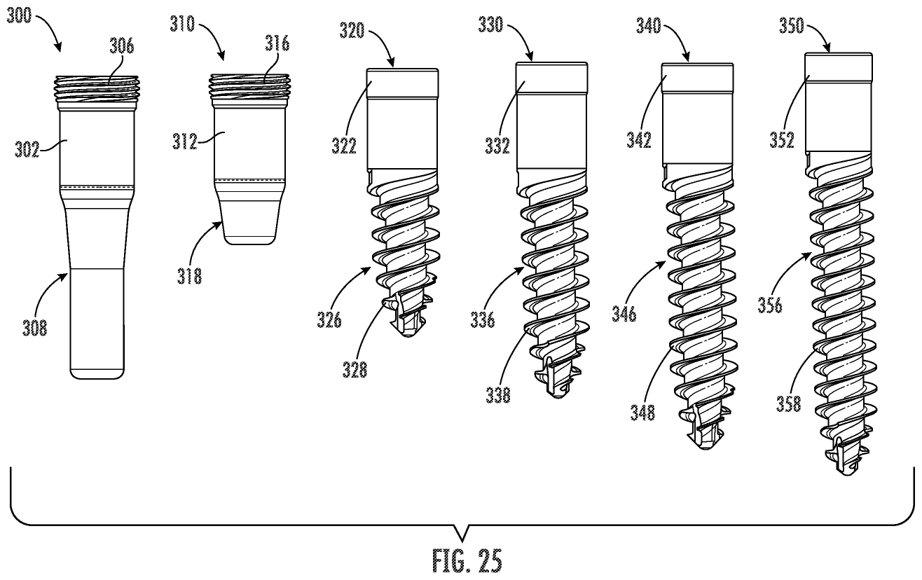

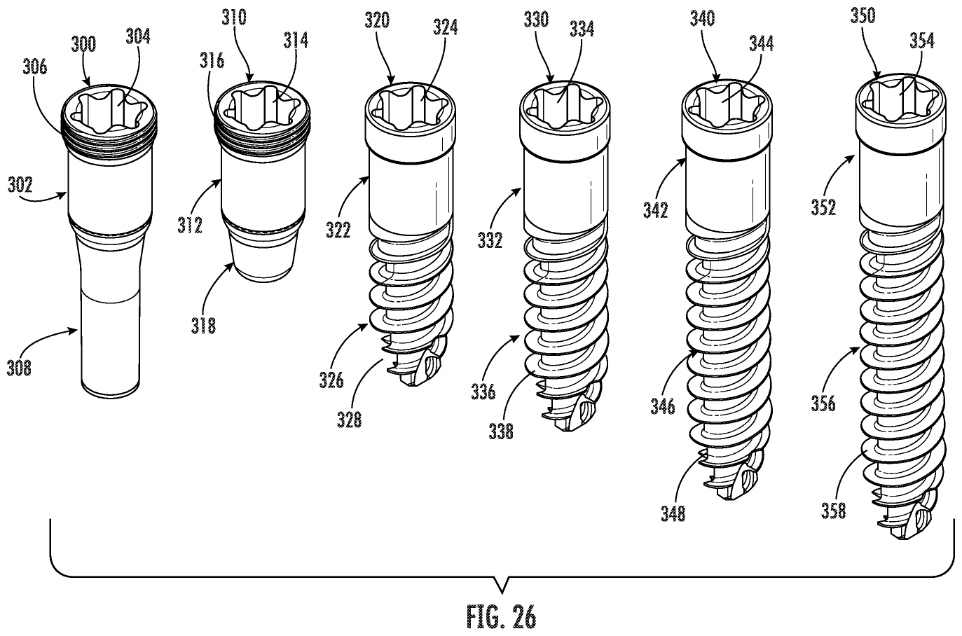

Referring now to FIGS. 25-26, the posts 300, 310 and screws 320, 330, 340, 350 are shown. The post 300 includes a head 302 and a stem 308 extending away from the head 302. The head 302 includes an opening 304 extending into the post 300 from a top surface toward the stem 308. The opening 304 may include a drive feature for engaging a tool for insertion of the post 300 through the baseplate 200. The head 302 also includes threads 306 extending around a portion of the head 302. The stem 308 may have, for example, a porous structure along at least a portion of the length allowing for bone ingrowth. The porous structure may be, for example, 3D printed. The porous structure may be, for example, a porous metal structure or porous coating. The stem 308 may have a diameter smaller than the diameter of the head 302. The post 300 may be tapered between the head 302 and stem 308.

The post 310 includes a head 312 and a stem 318 extending away from the head 312. The head 312 includes an opening 314 extending into the post 310 from a top surface toward the stem 318. The opening 314 may include a drive feature for engaging a tool for insertion of the post 310 through the baseplate 200. The head 312 also includes threads 316 extending around a portion of the head 312. The stem 318 may have, for example, a porous structure along at least a portion of the length allowing for bone ingrowth. The porous structure may be, for example, 3D printed. The porous structure may be, for example, a porous metal structure or porous coating. The stem 318 may have a diameter smaller than the diameter of the head 312. The post 310 may be tapered between the head 312 and stem 318. The stem 318 of the post 310 may be, for example, shorter than the stem 308 of the post 300.

The first screw 320 includes a head 322 and a stem 326 extending away from the head 322. The head 322 includes an opening 324 extending into the first screw 320 from a top surface toward the stem 326. The opening 324 may include a drive feature for engaging a tool for insertion of the first screw 320 through the baseplate 200. The head 322 may include a rim extending around the top of the head 322. The stem 326 may have, for example, threads 328 extending along at least a portion of the length. The stem 326 may taper from the head 322 to the distal end of the first screw 320.

The second screw 330 includes a head 332 and a stem 336 extending away from the head 332. The head 332 includes an opening 334 extending into the second screw 330 from a top surface toward the stem 336. The opening 334 may include a drive feature for engaging a tool for insertion of the second screw 330 through the baseplate 200. The head 332 may include a rim extending around the top of the head 332. The stem 336 may have, for example, threads 338 extending along at least a portion of the length. The stem 336 may taper from the head 332 to the distal end of the second screw 330.

The third screw 340 includes a head 342 and a stem 346 extending away from the head 342. The head 342 includes an opening 344 extending into the third screw 340 from a top surface toward the stem 346. The opening 344 may include a drive feature for engaging a tool for insertion of the third screw 340 through the baseplate 200. The head 342 may include a rim extending around the top of the head 342. The stem 346 may have, for example, threads 348 extending along at least a portion of the length. The stem 346 may taper from the head 342 to the distal end of the third screw 340.

The fourth screw 350 includes a head 352 and a stem 356 extending away from the head 352. The head 352 includes an opening 354 extending into the fourth screw 350 from a top surface toward the stem 356. The opening 354 may include a drive feature for engaging a tool for insertion of the fourth screw 350 through the baseplate 200. The head 352 may include a rim extending around the top of the head 352. The stem 356 may have, for example, threads 358 extending along at least a portion of the length. The stem 356 may taper from the head 352 to the distal end of the fourth screw 350.

The first screw 320 may be, for example, shorter than the second screw 330, third screw 340, and fourth screw 350. The second screw 330 may be, for example, shorter than the third screw 340 and fourth screw 350. The third screw may be, for example, shorter than the fourth screw 350. The fourth screw 350 may be, for example, longer than the third screw 340, second screw 330, and first screw 320.

In addition to the baseplates 100, 200, 250, the implant systems may include a locking screw 400 for coupling the baseplate 100, 200, 250 to a glenosphere. Referring now to FIGS. 27-30, the locking screw 400 is shown. The locking screw 400 includes a head 402 at a first end and a shaft 408 extending away from the head 402 to the second end. The head 402 includes an engagement opening 404 extending into the first end of the head 402. The engagement opening 404 is configured or sized and shaped to receive a tool for rotating the locking screw 400. The locking screw 400 may also include threads 406 extending around at least a portion of the exterior surface of the head 402. The threads 406 may be configured or sized and shaped to engage threads in the central opening 108, 202.

The shaft 408 may include a first non-threaded portion 410, a threaded portion 412, and a second non-threaded portion 414, as shown in FIGS. 28-30. The first non-threaded portion 410 couples to and extends from the head 402 toward the second end of the locking screw 400. The threaded portion 412 is positioned between the first non-threaded portion 410 and the second non-threaded portion 414. The second non-threaded portion 414 extends from the second end of the locking screw 400 to the threaded portion 412. The threaded portion 412 may be, for example, positioned near a midpoint of the shaft 408. The threaded portion 412 may be, for example, positioned between the first non-threaded portion 410 and the second non-threaded portion 414. The first and second non-threaded portions 410, 414 may be, for example, the same or different lengths. The threaded portion 412 may, for example, have a length the same size or smaller than the first and second non-threaded portions 410, 414.

The implant systems may also include a glenosphere 420 and at least one bone screw 430. The glenosphere 420 may include an opening 422 for receiving and coupling to the locking screw 400 to secure the glenosphere 420 to the baseplates 100, 200, 250. The bone screws 430 may be, for example, inserted through the screw holes 122 in the bone plates 100, 200, 250 to secure the bone plates 100, 200, 250 to the patient's bone.

With reference to FIGS. 27-30, the modular implant system may be assembled by inserting a post 300, 310 or screw 320, 330, 340, 350 into the central opening 202 of the plate 201, 252 of baseplates 200, 250. Next, a locking screw 400 may be inserted into the central opening 202 and the threaded portion 412 may rotatably engage the threads 208 of the second portion 206 of the central opening 202. Although not shown, in some embodiments, the locking screw 400 may be pre-assembled with a glenosphere, such that when the locking screw 400 is inserted into the baseplate 200, 250, the glenosphere is also coupled to the baseplate 200, 250.

As may be recognized by those of ordinary skill in the art based on the teachings herein, numerous changes and modifications may be made to the above-described and other embodiments of the present disclosure without departing from the scope of the disclosure. The components of the implants, devices, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants, devices, and/or systems may include more or fewer components or features than the embodiments as described and illustrated herein. For example, the components and features of implants 100, 200, 250 may be used interchangeably and in alternative combinations as would be modified or altered by one of skill in the art. Further, the steps of the surgical methods associated with the implants 100, 200, 250 may be used interchangeably and in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

1. A glenoid implant for implantation into a glenoid of a patient during an orthopaedic surgical procedure, comprising:
a baseplate comprising a plate with a top surface and a bottom surface, the plate having: (i) a central opening extending into the plate from the top surface, and (ii) a peripheral ring extending downwardly away from, and circumferentially around, the bottom surface, wherein (a) the peripheral ring has an interior surface and an opposite exterior surface, and (b) the exterior surface of the peripheral ring has a number of threads formed therein, and
a shaft coupled to and extending downwardly away from the bottom surface of the plate, the shaft having a number of threads formed in an outer surface thereof.

2. The glenoid implant of claim 1, wherein the threads formed in the exterior surface of the peripheral ring and the threads formed in the outer surface of the shaft have a common thread pitch.

3. The glenoid implant of claim 1, wherein the peripheral ring comprises a plurality of separate arcuate-shaped ring portions separated from one another by a gap.

4. The glenoid implant of claim 1, wherein the interior and exterior surfaces of the peripheral ring have a porous structure disposed thereon.

5. The glenoid implant of claim 1, wherein the shaft is integral with the plate.

6. The glenoid implant of claim 1, further comprising a locking screw and a glenosphere, wherein:
the central opening of the plate has a number of threads formed therein,
the glenosphere has a number of threads formed therein, and
the locking screw has a first number of threads configured to engage the threads of the central opening and a second number of threads configured to engage the threads of the glenosphere.

7. A glenoid implant for implantation into a glenoid of a patient during an orthopaedic surgical procedure, comprising:
a baseplate comprising a plate with a top surface and a bottom surface, the plate having: (i) a central opening extending into the plate from the top surface, and (ii) a peripheral ring extending downwardly away from, and circumferentially around, the bottom surface, wherein the peripheral ring has an exterior surface having a number of threads formed therein, and
a shaft coupled to and extending downwardly away from the bottom surface of the plate, the shaft having a number of threads formed in an outer surface thereof, wherein the threads formed in the outer surface of the shaft and the threads formed in the exterior surface of the peripheral ring have a common thread pitch.

8. The glenoid implant of claim 7, wherein the peripheral ring comprises a plurality of separate arcuate-shaped ring portions separated from one another by a gap.

9. The glenoid implant of claim 7, wherein the exterior surface of the peripheral ring has a porous structure disposed thereon.

10. The glenoid implant of claim 7, wherein the shaft is integral with the plate.

11. The glenoid implant of claim 7, further comprising a locking screw and a glenosphere, wherein:
the central opening of the plate has a number of threads formed therein,
the glenosphere has a number of threads formed therein, and
the locking screw has a first number of threads configured to engage the threads of the central opening and a second number of threads configured to engage the threads of the glenosphere.

12. A glenoid implant for implantation into a glenoid of a patient during an orthopaedic surgical procedure, comprising:
a baseplate comprising a plate with a top surface and a bottom surface, the plate having: (i) a central opening extending into the plate from the top surface, the central opening of the plate having a number of threads formed therein, and (ii) a peripheral ring extending downwardly away from, and circumferentially around, the bottom surface, wherein the peripheral ring has an exterior surface having a number of threads formed therein,
a shaft coupled to and extending downwardly away from the bottom surface of the plate, the shaft having a number of threads formed in an outer surface thereof,
a glenosphere having a number of threads formed therein, and
a locking screw having (i) a first number of threads configured to engage the threads of the central opening and (ii) a second number of threads configured to engage the threads of the glenosphere.

13. The glenoid implant of claim 12, wherein the threads formed in the outer surface of the shaft and the threads formed in the exterior surface of the peripheral ring have a common thread pitch.

14. The glenoid implant of claim 12, wherein the peripheral ring comprises a plurality of separate arcuate-shaped ring portions separated from one another by a gap.

15. The glenoid implant of claim 12, wherein the exterior surface of the peripheral ring has a porous structure disposed thereon.

16. The glenoid implant of claim 12, wherein the shaft is integral with the plate.
